(19) United States
(12) Patent Application Publication
(10) Pub. No.: US 2003/0109816 A1
(43) Pub. Date: Jun. 12, 2003

(54) Warmable bandage for promoting bandage for promoting wound healing

(76) Inventors: Charles A. Lachenbruch, Summerville, SC (US); Richard I. Barnett, Georgetown, SC (US)

Correspondence Address: KATHLEEN M HARLESTON, THE HARLESTON LAW FIRM, 909 TALL PINE ROAD, MT PLEASANT, SC, 29464

(21) Appl. No.: 10/013,419

(22) Filed: Dec. 8, 2001

(51) Int. Cl. A61F 5/00

(52) U.S. Cl. 602/2

(57) ABSTRACT

A warmable bandage for promoting wound healing includes: (a) phase change material having a melting point of between about 42 and 65 degrees Centigrade; (b) a gel or viscous fluid carrier in which the phase change material is substantially evenly distributed; (c) a fluid-impermeable, conformable envelope surrounding the phase change material and the gel or fluid carrier; and (d) at least one layer of thermal insulation covering an upper, upper surface of the envelope. A system and method for warming a warmable bandage are also included herein.

### BACKGROUND OF THE INVENTION

1. Technical Field

The present invention relates to a warmable bandage for warming a wound site to promote healing of the wound.

2. Background Information

The normal core temperature (“normothermia”) of the human body is between about 36° and 38° C. Skin temperature typically ranges between about 31° C. and about 38° C., depending on ambient temperature, the amount and type of clothing being worn, the core temperature, and where the skin is located on the body. Warming a wound site to a temperature within a specific temperature range that is slightly higher than body temperature promotes healing of chronic wounds. Vascularization of the subject area is also a factor, since a wound on skin that is less vascularized will heal more slowly. Warming the wound site is especially beneficial, sometimes critical, for diabetics and other individuals for whom wound healing is a problem. The problem of wound healing is also critical for geriatric patients, where suppurating sores or ulcers can become gangrenous and result in loss of a limb.

Several types of wound warmers (radiative heat) are available on the market, but they are oftentimes complicated to use. These conventional devices generally require electrical or other power for warming, and include control circuitry to maintain the desired temperature. Although they are used in some hospitals, they can be prohibitively expensive for an individual to own. Unfortunately, the majority of chronic wounds occur in settings in which expensive medical solutions are not an option. Demographic trends suggest that this situation will be magnified over the next several years.

A need for an inexpensive, easy to use warmable bandage for warming a wound site in order to speed healing was identified. This need has been addressed by the present invention, a multi-layered bandage, or wound covering, which can be heated in a conventional or special microwave and then placed over a wound. This inexpensive, easy to use bandage maintains a temperature within a prescribed range for a sufficient period of time to promote healing, when used in the prescribed manner. The bandage itself is not powered by electricity. At least one layer of the bandage is impregnated with phase change materials, preferably encapsulated, having a phase change such that heat will be delivered to the skin at between 37° and 43° C. Various phase change material technologies have been patented.

The bandage of the present invention can be warmed in the microwave or the like immediately before use, and then applied to the wound site (conductive heating). The bandage of the present invention emits a controlled amount of heat to the wound site, within the required temperature range, for a specific period of time (e.g., between about one and three hours). After that, the bandage of the present invention can be removed and discarded, or cleaned and reused later. These bandages can be placed over wounds or dressings that contain medications or compounds on the wound, and do not interfere with their effectiveness. They preferably include an adhesive on parts of the lower surface of the bandage for retaining the bandage on the patient's periwound skin area.

### BRIEF SUMMARY OF THE INVENTION

The present invention is a warmable bandage for treating wounds, which includes:

(a) phase change material having a melting point of between about 42 and 65 degrees Centigrade;

(b) a phase change material-compatible gel or viscous fluid carrier in which the phase change material is substantially evenly distributed;

(c) a fluid-impermeable, conformable envelope surrounding the phase change material and the gel or fluid carrier; and

(d) at least one layer of thermal insulation covering an upper surface of the envelope. There may also be a conformable, insulative layer between the PCM-containing envelope and the wound. A system and method for warming a warmable bandage are also included herein.

The warming bandages of the present invention are inexpensive and easy to use, and are therefore available for a wider range of individuals. They are inexpensive in comparison to other solutions because they do not require an electricity source or other source of power, nor do they require a microprocessor or control circuitry to maintain the therapeutic temperature. The appropriate temperature is delivered consistently, in part due to the thermal properties of the phase change compounds involved. The warming bandages of the present invention enable a healthcare provider to reduce costs because they need not purchase a separate, expensive warming unit, or pay care givers to locate the warming unit and transport it to the particular patient's bedside. These bandages are particularly useful for the elderly, and affordable even for small nursing homes. Other advantages include allowing individual patients or family members to provide their own care, thereby reducing costs and increasing independence. These bandages are customizable for different types of wounds on different areas of the body. They are made in different shapes and with different heating retention capacities. Finally, these bandages act as a physical barrier, protecting the wound site from outside contamination, and the patient and others from exposure to bacteria or other contaminants in the wound.

### BRIEF DESCRIPTION OF THE SEVERAL VIEWS OF THE DRAWINGS

A more complete understanding of the invention and its advantages will be apparent from the following detailed description taken in conjunction with the accompanying drawings, wherein examples of the invention are shown, and wherein:

FIG. 1 shows a perspective view of a warmable bandage according to the present invention;

FIG. 2 is a cross-sectional view of a warmable bandage according to FIG. 1, taken at line 2-2;

FIG. 3 is a perspective view of an alternate embodiment of a warmable bandage according to the present invention;

FIG. 4 is a cutaway view of a warmable bandage according to the present invention;

FIG. 5 is a top perspective view of a warmable bandage according to FIG. 1, shown in an outer wrapper;

FIG. 6 is a cross-sectional view of an alternate embodiment of a warmable bandage according to the present invention; and

FIG. 7 is a schematic view of a system of heating a warmable bandage according to the present invention.

### DETAILED DESCRIPTION OF THE INVENTION

In the following description, like reference characters designate like or corresponding parts throughout the several views. Also, in the following description, it is to be understood that such terms as “front,” “back,” “within,” and the like are words of convenience and are not to be construed as limiting terms. Referring in more detail to the drawings, the invention will now be described.

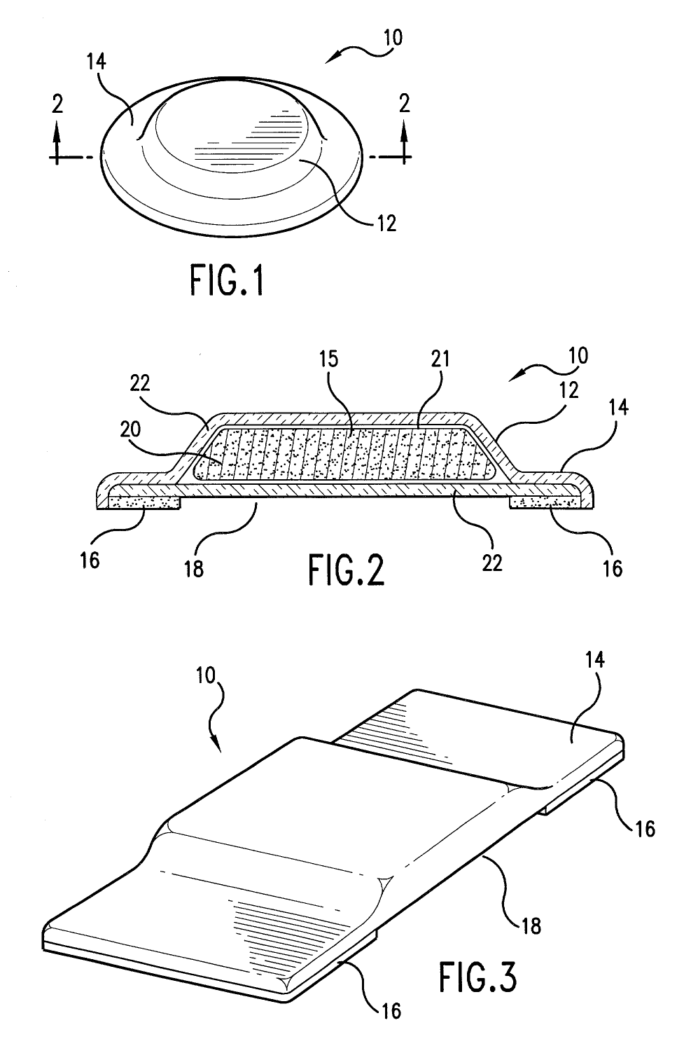

Turning first to FIG. 1, a preferred embodiment of the warmable bandage of the present invention, generally referred to as 10, is round with a central dome 12 on its upper surface 14. This shape is particularly well-suited for application on wounds on a patient's heel, elbow, and similarly shaped areas of the body. This bandage has alternate embodiments suitable for covering different shaped wounds on different areas of the body. This versatility is advantageous in that a warmable bandage which will remain on the wound will act as a consistent heat source for the wound. If the bandage is not constructed properly, it is liable to fall off, particularly where it is placed on angled or curved parts of the body. Also, an improperly constructed bandage is unlikely to apply sufficient heat at the appropriate temperature for the time interval necessary to bring about a therapeutic improvement. The bandage 10 comprises a phase change material 15 having a melting point, or phase change temperature, of between about 42 and 65 degrees Centigrade. The phase change material is one that is capable of being suspended in the gel or fluid carrier herein.

Referring to FIGS. 1 and 2, the dome 12 concentrates the heated area of the bandage over the wound, while nonabrasive, removable adhesive 16 is positioned on the lower surface 18 of the bandage, preferably along the thinner, peripheral edge of the bandage. The cross-section of FIG. 2 shows the inside of the warmable bandage 10. The center of the dome 12 holds a gel or viscous fluid carrier 20, such as a urethane gel or an oil, in which the phase change material 15 is substantially evenly distributed. It is believed that a gel is optimal for maintaining phase change material distribution and for even heating. The carrier and phase change material are both preferably non-toxic. In regard to materials intended to be in contact with the skin, two important parameters for obtaining FDA (Food and Drug Administration) approval are skin irritability and cytotoxicity. Another consideration is the need for the carrier to absorb microwave energy. Urethane gels and mineral oils are among the suitable gel and fluid carriers in this regard. It is also believed that inexpensive oils, such as vegetable oil, olive oil, or peanut oil, may also be employed herein, so long as they have a suitably high viscosity and high specific heat and a preservative is included. The viscous, conformable fluid herein has a viscosity greater than about five centipoise, and it should have high heat capacity, be unattractive to bacteria or fungus when confined in the air- and fluid-impermeable envelope, and be capable of absorbing large amounts of microwave radiation.

The phase change material is preferably microencapsulated so that it remains evenly distributed throughout the gel or fluid carrier even after heating and cooling. This is important because many phase change materials, such as the alkanes, are by themselves poor thermal conductors, and in the pure form, are hard, rigid compounds in the solid state that cannot be conformed to the curves of the body. The distribution of the phase change material into small, generally spherical capsules with a diameter of between about one and 100 microns significantly enhances heat transfer between the surrounding medium and the phase change material. Microencapsulation also prevents interaction, chemical or otherwise, over time between the phase change material and the carrier or envelope material, thus increasing product durability. Any suitable method for encapsulating the phase change material in a protective coating can be utilized. Powder phase change material is preferably used because it is believed to have good conductivity due to its higher surface area. The phase change materials are preferably microencapsulated in a thin coating, more preferably a polymer. The coating preferably forms a generally spherical shell around the phase change material with a shell thickness of between about 0.03 and two microns, most preferably about 0.05 micron thick.

As shown in FIGS. 1, and 2, the PCM-containing fluid or gel 20 is contained within a thin, fluid impermeable, conformable envelope 21, which surrounds the PCM-containing gel or fluid. For an average sized, rectangular shaped bandage of between about two and five inches wide and about three and six inches long, the envelope preferably contains between about 50 and about 250 grams of the phase change material and between about 30 and 150 grams of the gel/fluid carrier. The concentration of phase change material to coating material by weight is preferably between about 1:4 and 4:1, most preferably about 50:50. This ratio has generally been found to be of importance because, for most types of phase change materials and carriers, the more phase change material that is added to the carrier beyond a certain minimal level, the stiffer and firmer the mixture becomes. Although in general more phase change material means better and longer warming, too much phase change material results in an overly bulky, unacceptably hard bandage. Only a small amount of PCM/carrier is required in each bandage to achieve the desired therapeutic effect, so the bandage of the present invention can have a polished, sleek look.

The envelope 21 is preferably made of a urethane film with a thickness of between about 0.003 and 0.018 inch. The envelope 21 is preferably lenticular, or elliptical, in shape, resembling a partially deflated balloon. For broader wounds such as burns, the envelope may be doughnut-shaped so that growth along the shallower edges of the wound is fostered. With the doughnut (ring) shape, heat is not uncomfortably expended over the central part of the wound, where more skin layers may have been burned through. Thus, the shape and size of the envelope and the bandage may be varied according to the type of wound. Since wounds vary widely in terms of depth and breadth, how clean the edges of the wound are, how the skin has been compromised, the likelihood that the wound has been exposed to infection, etc., a customizable bandage is desirable and can quicken the healing process.

As shown in FIGS. 1, 2, and 4, the PCM/gel-containing envelope 21 is surrounded by a conformable thermal insulation layer 22, which is seen on the upper and lower surfaces of the bandage 10 in FIG. 2. This insulation layer 22 is preferably made of thin sheets of hollow fiber polymer, or any other suitable insulative, cushioning, comfortable, absorbent, conformable material, which provides bulk and softness. The primary function of the insulation layer is to limit the escape of heat from the envelope. The insulation layer may be thin or thick. It covers the upper surface of the envelope 21, and, in some embodiments, the bottom of the envelope.

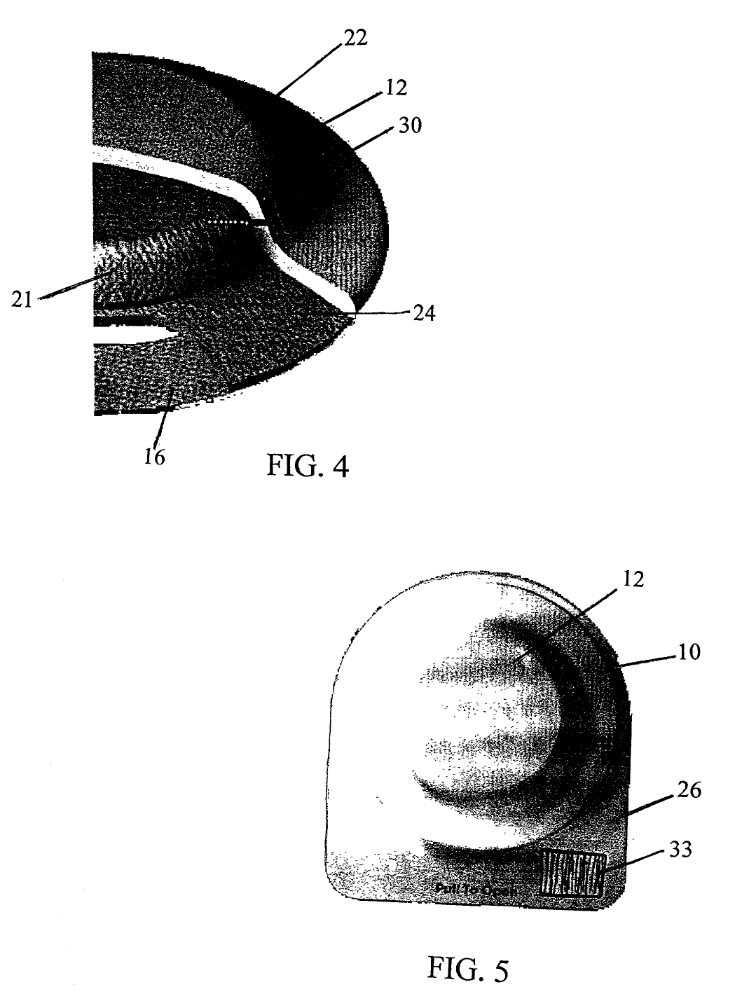

As shown in FIGS. 2 and 4, the circular bandage 10 preferably includes a first insulation layer 22 over the upper surface of the bandage, and an additional, second layer 24 of the same or different insulation material extending under the elliptical PCM-containing envelope 21 at the base of the bandage. The second insulation layer 24 can be made of the same or different material than the first insulation layer 22, but it should not stick to the wound. In some embodiments, this second insulation layer may not be present under the PCM-containing envelope. Around the periphery of the bandage, the second insulation layer 24 is sandwiched between the lower adhesive contact material 16 and the upper insulation layer 22. As shown in FIG. 2, the first insulation layer 22 extends over the outer edge of the adhesive 16. In a preferred embodiment, then, a generally circular upper layer 22 of the insulation extends over the envelope 21, which is generally elliptical in shape, and a generally circular second layer 24 of the insulation extends below the envelope 21; the periphery of the second insulation layer 24 being sandwiched between the upper first insulation layer 22 and a ring of the adhesive 16; the ring of adhesive 16 extending around the periphery of the generally round bandage 10 between an outer edge of the envelope 21 and an outer edge of the bandage 10. Bandages herein may also have a water repellent coating.

Referring to FIGS. 2 and 4, the adhesive 16 is in a continuous band around the periphery of the lower surface 18 (see FIG. 4), or segments of adhesive can be spaced along the periphery of the lower surface 18. The adhesive 16 temporarily attaches the warmable bandage 10 to the skin around the wound site. The phrase “wound site” herein is meant to include the wound and the adjacent periwound area. The word “wound” here refers to surgical incisions, post-amputation sites, bums, ulcers (such as venous ulcers, and diabetic ulcers), sores (such as pressure ulcers), or other lesions or breaks in the skin that are open to the surface.

Any suitable adhesive may be used, so long as it is easy to remove and does not harm the body. Pressure sensitive adhesives are preferred, particularly water-based or solvent-based adhesives, such as thermoplastic polymers, applied to a suitable backing material. Radiation-cured pressure sensitive adhesives, such as UV-curable films, may also be suitable for use herein.

Although the warmable bandage is preferably disposable, a less preferred embodiment is autoclavable and usable over disposable dressings. A medicinal gel or other compound is preferably applied to the wound prior to placement of the bandage over the wound. The medicinal gel keeps the wound moist, so the bandage is less likely to stick to the wound, and preferably contains an antibiotic to prohibit bacterial growth in the wound.

Turning now to FIG. 3, an alternate embodiment of the warmable bandage 10 is rectangular in shape, although its cross-section resembles FIG. 2 in appearance. This rectangular embodiment is particularly well-suited for long, curved extremities, such as the arms and legs. The adhesive 16 appears on either end of the bandage. Again, the bandage is thicker in the middle to accommodate the PCM/gel, and hold the heat over the wound.

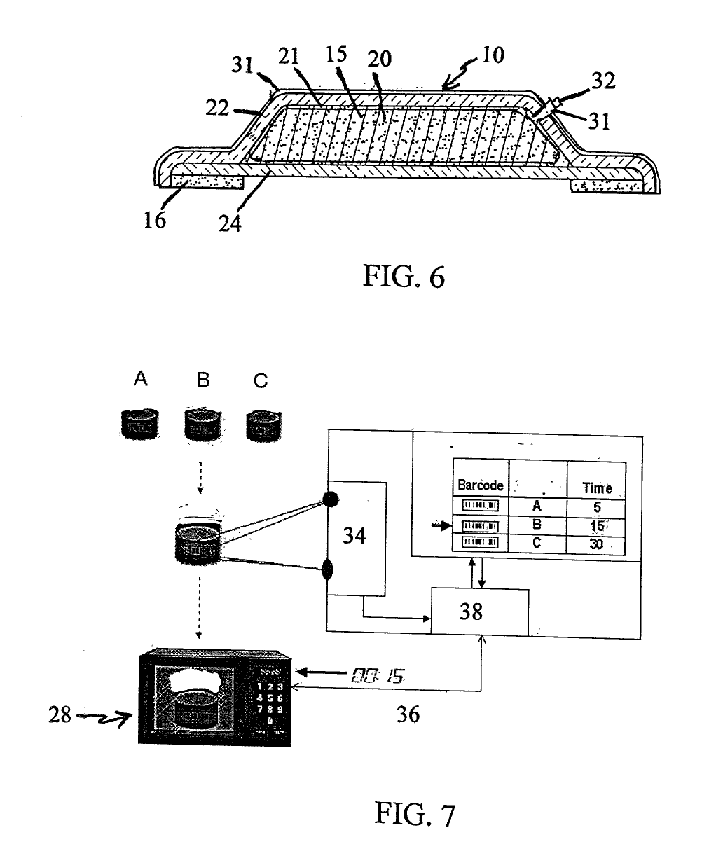

As shown in an alternate embodiment in FIG. 6, the bandage 10 an outer protective covering 25, preferably urethane film, over the insulation layer 22 on the upper surface 14 of the bandage 10. The outer covering 25 may be ventilated, and it may be impregnated with an antibiotic substance in order to discourage bacterial growth in the area. This outer layer provides integrity, keeps the wound and bandage clean, and reduces the likelihood that the bandage will be punctured. Also, the outer covering 25, along with the outer insulation layer, largely prevents heat from the envelope 21 from escaping through the upper surface 14 of the bandage 10. Heat is instead directed down through the lower surface 18 onto the wound. Even immediately after heating, then, the upper surface 14 of the bandage 10 will not be hot or warm to the touch. The lower surface 18 of the bandage 10 will also not be hot to the touch, preferably no more than about 45 degrees Centigrade. The warmed bandage will therefore not be dangerous to either the caregiver or the patient.

Referring to FIGS. 4, 5, and 6, the warmable bandage 10 is enclosed in an outer wrapper 26, as shown in FIG. 5, which keeps the bandage clean prior to use. Immediately prior to using the warmable bandage 10, a corner of the wrapper 26 is loosened, per a set of instructions (not shown) on the back of the wrapper, and the bandage in its wrapper is placed in a microwave 28 for heating. It is important that the bandage not be heated above the recommended temperature range because it could conceivably harm a patient.

Various systems can be used for determining when the warmable bandage 10 is heated to an appropriate degree. One system includes a small thermal window 30 or slit on the upper surface 14 of the bandage 10. In FIG. 4, a color-coded window 30 is shown on the dome 12, where it is visible to the user looking in through the front window of the microwave 28. The thermal window 30 allows the user to view a thermal indicator placed inside the bandage, which senses the internal temperature of the bandage. The thermal indicator changes in some visible way, such as a color change, to indicate to the caregiver or user that the PCM-containing gel or fluid carrier has been heated to the pre-determined temperature. This system is inexpensive because a home microwave can be employed to heat bandages for the patient. A color-coded temperature indicator may also be applied directly on the upper or lower surface 14, 18 of the bandage 10.

Referring to FIG. 6, another way for the user to determine when the bandage is properly heated is a pop-up thermometer 31. The bandage 10 is purchased with a popup thermometer 31 inserted in the dome 12. The lower end of the thermometer rests inside the bandage, preferably in, on, or very near the envelope 21 that encloses the PCM-containing gel or fluid 20. When the pre-determined temperature is reached, a brightly colored tab 32 on the upper end of the thermometer 31 pops out, signaling to the user that the bandage 10 is heated and can be removed from the microwave, and applied to the wound.

Another system for properly heating the bandage 10 involves a temperature bar code 33 on the bandage wrapper 26, as shown in FIG. 5. The special microwave 28, which is shown in FIG. 7, is outfitted with a bandage identifier, such as a bar code reader 34 or a similar package identifier. The bandage identifier identifies the bandage and inputs to the microwave regarding the length of time the microwave is on. The coded, predetermined time is based on how long it takes for approximately ¾ of the PCM to reach its phase change temperature when packaged as identified by the code. That is, the microwave oven is timed to warm the carrier and melt approximately 3/4of the phase change material. The bar code (or similar) reader reads the temperature bar code 33 and initiates a timed heating cycle at a pre-programmed power setting. The microwave 28 can be programmed for more than one temperature setting or period of time.

Without meaning to be bound by theory, it is believed that the fact that about ¼ of the phase change material is left unmelted is the safety factor that ensures that the bandage 10 will not be overheated. As long as some phase change material remains in the solid state, the temperature of the envelope will not exceed its phase change temperature.

The bandage of the present invention preferably does not comprise a power source; since it is pre-heated, it does not have to be attached to an electrical source or batteries while it is on the user, nor is control circuitry necessary, in order for the bandage to maintain heat for the required therapeutic period of time. The bandage 10 does not include a microprocessor, EEPROM, or the like.

FIG. 7 shows a microwave 28 that has been programmed for three settings triggered by three different bar codes, for example. In this example, the first bar code on the first bandage A initiates a five second timing interval, while the second bar code on B initiates a 15 second timed sequence, and the third bar code on C codes for a 30 second sequence. The different times are required to warm the particular quantity of carrier in the specific bandage, as well as to melt, or “thermally charge”, a high percentage of phase change material 15 in the envelope 21, preferably between about 70 and 85% of the phase change material. Without meaning to be bound by theory, it is believed that it is important not to overheat the bandage because as soon as 100% of the phase change material is melted, the temperature control behavior provided by the phase change is lost. If the phase change material were fully melted, the bandage could be heated to temperatures which can harm the wound area and surrounding skin.

Wound treatment research may prove that different temperatures are advantageous for different types of wounds, or that varying the temperature over the course of treatment of a single wound, such as a diabetic ulcer, helps. The identifier would allow for customizing warming cycles for bandages that are custom-built for different wounds. Skin temperatures vary as much as ten degrees across the human body. The extremities normally have a lower skin temperature than the skin temperature of the torso, for example, and wounds vary in severity (e.g., depth of penetration) and type, so different shapes, types, and melting temperatures are useful for various bandages herein. The bandage of the present invention is versatile and can be customized for different situations by varying the phase change material, type and amount of carrier(s), layers of insulation, and shape of the bandage. Generally, the more insulation the bandage has, the slower heat is released from the warmed bandage.

Continuing with FIG. 7, within the microwave 28, an interface cable 36 connects the front panel of the microwave to the bar code identifier 34. The bar code identifier 34 inputs to a microprocessor 38 (logic circuit) with memory. The microprocessor 38 starts the microwave 28 and stops it at the end of the pre-determined time period.

Thus, the present invention includes a system for warming a warmable bandage, comprising:

(a) a warmable bandage 10 in an outer wrapper 26;

(b) a warming cycle temperature bar code 33 or similar identifier on the bandage 10 or bandage wrapper 26; and

(c) a microwave 28 comprising a warming cycle temperature bar code identifier, a microprocessor 38, and memory;

wherein the bandage is warmable in the microwave at a pre-determined power setting for a pre-determined time interval coded by the temperature bar code.

Also included herein is a system for warming a warmable bandage, comprising:

(a) a warmable bandage 10 comprising a color coded thermal window 30; and

(b) a microwave 28;

wherein the thermal window 30 changes color when it reaches a predetermined temperature in the microwave.

Also included herein is a system for warming a warmable bandage, comprising:

(a) a warmable bandage 10 comprising phase change material contained in a conformable, fluid impervious envelope 21;

(b) a pop-up thermometer 31 in the bandage 10, a lower end of the thermometer 31 contacting the envelope 21; and

(c) a microwave 28;

wherein the bandage 10 is warmable in the microwave 28 at a pre-determined power setting, and a tab 32 on an opposite, upper end of the thermometer 31 pops up when a pre-determined temperature is reached by the contents of the envelope. An alternative temperature indicator may be suitable for use herein instead of the pop-up thermometer.

The type of phase change material utilized herein can be varied according to budget, temperature requirements, and length of time that heating is desired. Although the PCM-carrier mixture is expected to lose heat after a time (since this heat is transferred primarily to the wound), it has been found that a PCM-carrier mixture that remains heated above body temperature for longer than about 20 minutes is useful herein. Preferably, the phase change materials used herein melt at a temperature of between about 42 and 65 degrees Centigrade, more preferably between about 44 and 55, degrees Centigrade. The warm phase change material warms the carrier, which warms the urethane film envelope, which warms the insulation, which warms the patient's skin.

Preferably, once the phase change material 15 is suitably heated, the bandage 10 heats the skin around the user's wound site to between about 37 and 43 degrees Centigrade for between about 20 minutes and three hours, preferably between about 30 and 60 minutes. The temperature of the carrier and the phase change material suspended in it must be a few degrees warmer than this in order to drive heat toward the body. Some heat will be lost to the environment once the bandage is placed on the user's body.

Phase change materials are normally classified according to their melting points. Since most phase change materials are not pure, they melt over a range of several degrees of temperature. When they are heated to a temperature within this temperature range, the bulk of the phase change materials within the phase change material mixture will melt from a solid to a liquid. Many variables contribute to the performance of the bandages, including the type of phase change material, the mixture, the carrier, whether they are encapsulated, how long they are heated, the outer thermal layer ambient temperature, and the body temperature of the patient on whom the bandage is applied.

As the phase change material cools, it undergoes a phase change, which is how it provides heat to the wound below the bandage. Because this phase change occurs at a specific temperature, the heat can only be delivered at this specific temperature, or slightly less than it, depending upon the thickness of the insulation between the PCM-containing carrier and the wound site. This makes it ideal for temperature-controlled application of heat.

Of course, the heat conveying benefit of the bandage 10 is not continuous; heat is only generated for a limited time after the initial heating of the bandage. That length of time, though, is sufficient to provide a noticeable therapeutic benefit when these warmable bandages are applied to the wound site several times a day for several days.

The caregiver need not be concerned that the product will cool down too rapidly, or deliver its heat at any temperature other than the set temperature. The latent heat that is released by the solidifying phase change material in the bandage envelope can only be released at the specific set temperature. The type of phase change material and the number and type of insulation layers are selected to ensure that the temperature that reaches the skin is one that is not harmful, and in fact is known to convey a therapeutic benefit.

Also, the heat generated by the bandage is predictable and stable, so the caregiver need not be concerned about the product heating up, which would violate established laws of thermodynamics, or cooling down too rapidly (as is the case with other conventional methods for applying heat). Once the caregiver applies the warmable bandage 10, he or she is free to perform other tasks. Once it is heated, the bandage 10 itself, which is covered by the thermal insulation layer 22, is not too hot to handle.

Suitable phase change materials for use herein include C22 to C30 alkanes (i.e., alkanes with between about 22 and 30 carbons) or mixtures thereof, and acetate salts, preferably sodium acetate. Preferred alkanes for use herein are docosane (C22), tricosane (C23), tetracosane (C24), pentacosane (C25), hexacosane (C26), octacosane (C28), and triacontane (C30). Alkanes may also be selected and mixed based on budget constraints, since some of them are much more expensive than others. For example, a 24-carbon paraffin, tetracosane, is expensive. A less expensive alternative is a mixture of C23, C25, C26, and C27 alkanes, which has a peak melting point of 50 degrees C., but the peak of the melting curve for this mixture is broader and less precise than that of tetracosane in that melting actually occurs over a 47 to 51 degree C. range.

The present invention also includes a method for warming a warmable bandage, comprising the steps of:

(a) automatically reading a temperature warming cycle bar code, or similar identifier, on a warmable bandage comprising phase change material with a melting point between about 42 and 65 degrees Centigrade in a microwave, or on the bandage packaging, using a temperature warming cycle bar code identifier, or a similar identifier, in the microwave;

(b) automatically initiating warming of the warmable bandage in the microwave to a temperature of between about 42 and 65 degrees Centigrade;

(c) automatically stopping the microwave at the end of a predetermined, period of time coded by the temperature warming cycle bar code identifier; and

(d) removing the bandage from the microwave and applying it to a wound site. Prior to these steps, the user places the warmable bandage in the microwave and presses an “on” button. The method preferably further includes step (b1): heating between about 70 and 85% of the phase change material in the bandage to its melting point.

From the foregoing it can be realized that the described device of the present invention may be easily and conveniently utilized as a warmable bandage for promoting wound healing. It is to be understood that any dimensions given herein are illustrative, and are not meant to be limiting.

While preferred embodiments of the invention have been described using specific terms, this description is for illustrative purposes only. It will be apparent to those of ordinary skill in the art that various modifications, substitutions, omissions, and changes may be made without departing from the spirit or scope of the invention, and that such are intended to be within the scope of the present invention as defined by the following claims. It is intended that the doctrine of equivalents be relied upon to determine the fair scope of these claims in connection with any other person's product which fall outside the literal wording of these claims, but which in reality do not materially depart from this invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is:

1. A warmable bandage for promoting wound healing, the bandage comprising:
(a) phase change material having a melting point of between about 42 and 65 degrees Centigrade;
(b) a phase change material-compatible gel or viscous fluid carrier in which the phase change material is substantially evenly distributed;
(c) a fluid-impermeable, conformable envelope surrounding the phase change material and the gel or fluid carrier; and
(d) at least one layer of conformable, thermal insulation covering an upper, outer surface of the envelope.

2. A warmable bandage according to claim 1, wherein the phase change materials are microencapsulated in a protective coating, and the insulation layer surrounds the envelope.

3. A warmable bandage according to claim 2, which does not comprise a power source or microprocessor.

4. A warmable bandage according to claim 1, wherein the envelope is a urethane film.

5. A warmable bandage according to claim 4, wherein the phase change material is a C22 to C30 alkane, or a combination thereof.

6. A warmable bandage according to claim 5, further comprising a pressure sensitive adhesive on a lower surface of the warmable bandage.

7. A warmable bandage according to claim 6, wherein a generally circular upper, first layer of the insulation extends over the envelope, which is generally elliptical in shape, and a generally circular second layer of the insulation extends below the envelope; the periphery of the second insulation layer being sandwiched between the upper, first insulation layer and a ring of the adhesive; the ring of adhesive extending around the periphery of the generally circular bandage between an outer edge of the envelope and an outer edge of the bandage.

8. A warmable bandage according to claim 6, wherein the phase change material is a combination of C24 and C26 alkanes.

9. A warmable bandage according to claim 8, wherein the PCM-containing fluid is an oil.

10. A warmable bandage according to claim 8, wherein the thermal insulation layer comprises a hollow fiber polymer insulation.

11. A warmable bandage according to claim 10, wherein the PCM-containing fluid is mineral oil.

12. A warmable bandage according to claim 7, wherein the PCM-containing gel is urethane gel.

13. A warmable bandage according to claim 3, wherein the phase change material is an acetate salt.

14. A warmable bandage according to claim 9, further comprising a pop-up thermometer in the dome of the bandage.

15. A warmable bandage according to claim 9, wherein the phase change material is sodium acetate.

16. A warmable bandage according to claim 9, wherein the phase change material is encapsulated with polymer, forming generally spherical PCM/polymer microcapsules which range in diameter between about one and 100 microns.

17. A warmable bandage according to claim 9, further comprising an outer covering on the upper bandage surface of urethane film.

18. A warmable bandage according to claim 9, further comprising a color coded window on the outer surface of the bandage for signaling that the PCM-containing gel or fluid carrier has been heated to a pre-determined temperature.

19. A system for warming a warmable bandage, comprising:
(a) a warmable bandage in an outer wrapper;
(b) a warming cycle temperature bar code on the bandage or bandage wrapper; and
(c) a microwave comprising a warming cycle temperature bar code identifier, a microprocessor, and memory;
wherein the bandage is warmable in the microwave at a pre-determined power setting for a pre-determined time interval coded by the bar code.

20. A system for warming a warmable bandage, comprising:
(a) a warmable bandage comprising a color coded thermal window; and
(b) a microwave;
wherein the thermal window changes color when it reaches a predetermined temperature in the microwave.

21. A method for warming and using a warmable bandage, comprising the steps of:
(a) automatically reading a temperature warming cycle bar code on a warmable bandage comprising phase change material with a melting point between about 42 and 65 degrees Centigrade in a microwave, or on the bandage packaging, using a bar code identifier in the microwave;
(b) automatically initiating warming of the warmable bandage in the microwave to a temperature of between about 42 and 65 degrees Centigrade;
(c) automatically stopping the microwave at the end of a pre-determined, period of time coded by the temperature warming cycle bar code identifier; and
(d) removing the bandage from the microwave and applying the bandage to a wound site.

22. A warmable bandage according to claim 21, further comprising step (b1) heating between about 70 and 85% of the phase change material in the bandage to its melting point.
